(19) United States
(12) Patent Application Publication
(10) Pub. No.: US 2024/0269342 A1
(43) Pub. Date: Aug. 15, 2024

(54) GREEN SYNTHESIS OF Ag-Ni BIMETALLIC NANOPARTICLE-INCORPORATED PVA/PAA HYDROGELS WITH ANTIMICROBIAL PROPERTIES

(71) Applicant: Yuvraj Ruparel, Mumbai (IN)

(72) Inventor: Yuvraj Ruparel, Mumbai (IN)

(21) Appl. No.: 18/606,410

(22) Filed: Mar. 15, 2024

(30) Foreign Application Priority Data
May 19, 2023 (IN) 202321035118

(51) Int. Cl. A61L 26/00; B82Y 40/00

(52) U.S. Cl. CPC A61L 26/0004; A61L 26/0014; A61L 26/0066; A61L 26/008; A61L 2300/104; A61L 2400/12; B82Y 40/00

(57) ABSTRACT

Inert hydrogels play a crucial role in burn first aid. If there is no access to clean water or if the burns are severe, hydrogel dressings may be used to cool burn wounds instead of running water. This is especially helpful in situations when clean water is not available. Hydrogels that have a high percentage of water content can dissipate the heat that builds up on the skin. In addition to relieving the discomfort, this will keep the area clean and protected from any additional injury.

The manufacture of bimetallic Ag—Ni nanoparticles from a plant extract is at the core of the present invention. These nanoparticles are then incorporated into a PVA/AA hydrogel that is of a quality suitable for medical use. Since the nanoparticles in hydrogel possess antibacterial qualities, the combination may be used to speed up the healing process. The PVA/AA polymer of medical grade that was used in the production of the hydrogels does not irritate the skin and is not harmful to the environment. To produce Ni—Ag nanoparticles in a PVA/AA matrix, we employed one pot green synthesis method. For the purpose of assisting in the speedy recovery of burn wounds and providing protection against E. coli, they operate as antibacterial agents that are extremely effective due to the size of the particles, which is 12 nm.

### CROSS-REFERENCE TO RELATED APPLICATION(S)

This application claims priority to Indian Provisional Application No.: 202321035118, Filed on May 19, 2023, the disclosures of which are hereby incorporated by reference in their entirety.

### BACKGROUND OF THE INVENTION AND PRIOR ART

There are three advantages that come along with the use of hydrogel dressings for the treatment of burn wounds. Hydrogel dressings have the potential to absorb wound exudate first and foremost. It is possible for a hydrogel to have an absorption capacity that is hundreds of times higher than its dry weight. In addition, the capacity of hydrogels to disseminate fluids helps the preservation of a moist environment for wound healing, which is essential for the successful treatment of dry wounds. This benefit may be achieved by the use of hydrogels. Second, hydrogel dressings may be shaped into any configuration necessary, depending on the nature of the wound that has to be treated. Last but not least, hydrogel dressings could be able to alleviate some of the pain and warmth associated with the wound while still adhering to it without becoming stuck. In addition to this, they are transparent, which makes it possible to view the wound that has been caused. Research on hydrogel-based burn therapies is expanding in a number of countries throughout the world, and several discoveries as well as advancements have been made.

Since the creation of nanotechnology in the previous century, the majority of progress in nanomedicine, also known as the use of nanotechnology in medicine, has taken place during the last three decades. The use of nanoparticles (NPs) is widespread across a wide range of sectors, including the purification of water, the production of textiles, the development of biosensors, and the preservation of food. The primary function of NPs in the pharmaceutical and medical industries is that of an antibacterial agent. In addition to their ability to fight bacteria, NPs also possess antiviral, antifungal, anti-inflammatory, anti-angiogenic, anti-tumoral, and antioxidant activity. Moreover, NPs have the potential to serve as a delivery vehicle in the fields of imagcology and cosmetics. Antibacterial effectiveness may be affected by physicochemical features such as size, shape, dispersion, and concentration of the particles.

Because of the benefits that metal nanoparticles (such as silver, gold, and zinc) have on the healing of wounds, as well as the treatment and prevention of bacterial infections, dermatology is increasingly making use of these substances. Other benefits include the case of use, the reduction in the number of dressing changes required, and the maintenance of a moist environment around the wound. NPs have key characteristics that include their size and shape, which have a role in active substance transport (carrier circulation), penetrability (either directly through cell membranes or via phagocytosis), and cellular responses. Because of their antibacterial properties and low toxicity profiles, metal NPs including silver, gold, and zinc are good candidates for inclusion into wound dressings. The four most common types of nanomaterials used in wound care are nanoparticles, nanocomposites, coatings, and scaffolds.

Due to the significance that the scientific community places on this topic, a number of review articles that centre on the utilisation of NPs in conjunction with hydrogels have been released in the most recent few years. When NPs and hydrogel are combined, the hydrogel shields the NPs from degradation and prevents them from aggregating. It also increases the local distribution of medications and demonstrates great antibacterial activity, both of which aid in the speedy recovery of wounds. Incorporating NPs into hydrogels may, in a concentration-dependent relationship, result in improvements to the hydrogel's mechanical characteristics, such as its strength, stiffness, and degradation.

### BRIEF DESCRIPTION OF THE FIGURES

FIG. 1 shows PVA/PAA Hydrogel before nanoparticle incorporation.

FIG. 2 shows Coconut Coir Extract preparation.

FIG. 3 shows PVA/PAA Hydrogel after nanoparticle incorporation.

FIG. 4 shows Schematic illustration of Synthesis of Ag—N PVA/PAA hydrogel.

FIG. 5 shows FT-IR spectra of Ag—Ni-PVA/PAA hydrogel.

FIG. 6 shows Particle size measurement using DLS.

FIG. 7 shows Zeta potential measurement.

FIG. 8 shows Ag—Ni PVA/PAA hydrogel SEM image.

FIG. 9 shows Well diffusion method test image.

### DESCRIPTION OF THE INVENTION

### Synthesis of PVA/PAA Hydrogel

FIG. 1 shows PVA/PAA Hydrogel before nanoparticle incorporation.

To produce aqueous Poly vinyl alcohol (PVA), it was dissolved in ultra-pure water at 70° C. while being magnetically agitated. After cooling to room temperature, the mixture was added to a flask with three necks and a condenser. While being magnetically agitated, the required concentrations of acrylic acid (AA) monomer (5 mol % of AA monomer per vinyl alcohol repeating unit) were added to the PVA solution along with the addition of 1000 ppm ammonium persulfate as an initiator. The flask was sealed and set in an oil bath that had been heated to 80° C. after being argon-purged for 30 minutes while swirling the mixed solution. The impact continued for 48 hours after that. The solution was filtered and allowed to sit for the night in order to get rid of the undissolved particles and bubbles. After that, a homogeneous polymer solution was produced.

### Coconut Coir Extract Preparation

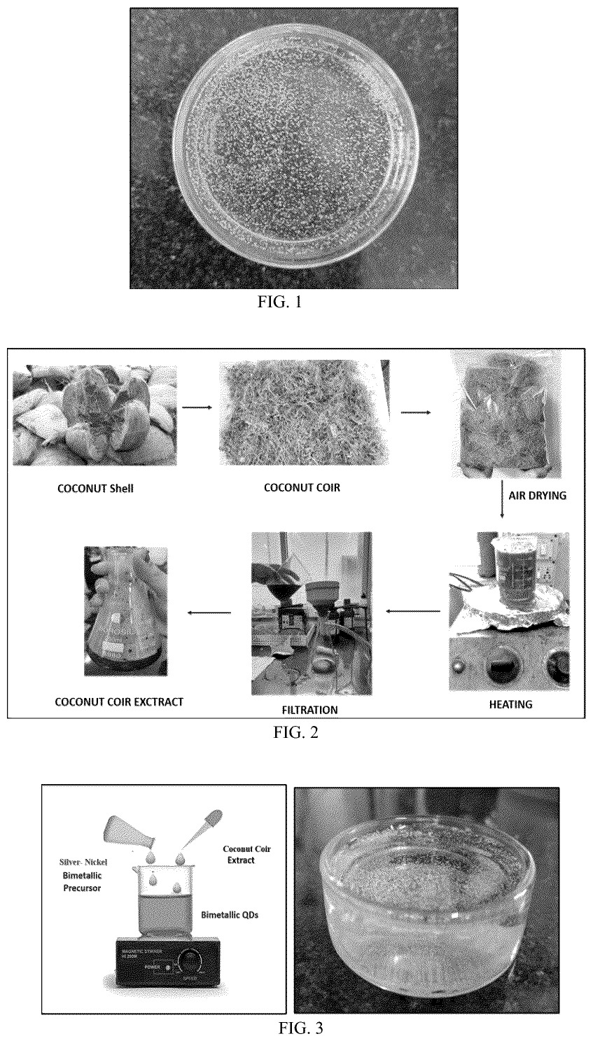

FIG. 2 shows Coconut Coir Extract preparation.

The dust was removed from the coconut coir by washing it with deionized water, and then allowing it to dry at room temperature (22-26° C.) for 24 hours before being powdered. An aqueous extract was made by combining 2 gm of powdered leaves with 60 ml of deionized water at a temperature of 70° C., stirring the mixture continuously for two hours, and then immediately filtering the mixture using Whatman filter paper. Before being used to synthesise NPs, the aqueous extract of coconut coir was kept at a temperature of 4° C.

### Synthesis of Bimetallic Ag—Ni Nps in Polymer Matrix

FIG. 3 shows PVA/PAA Hydrogel after nanoparticle incorporation.

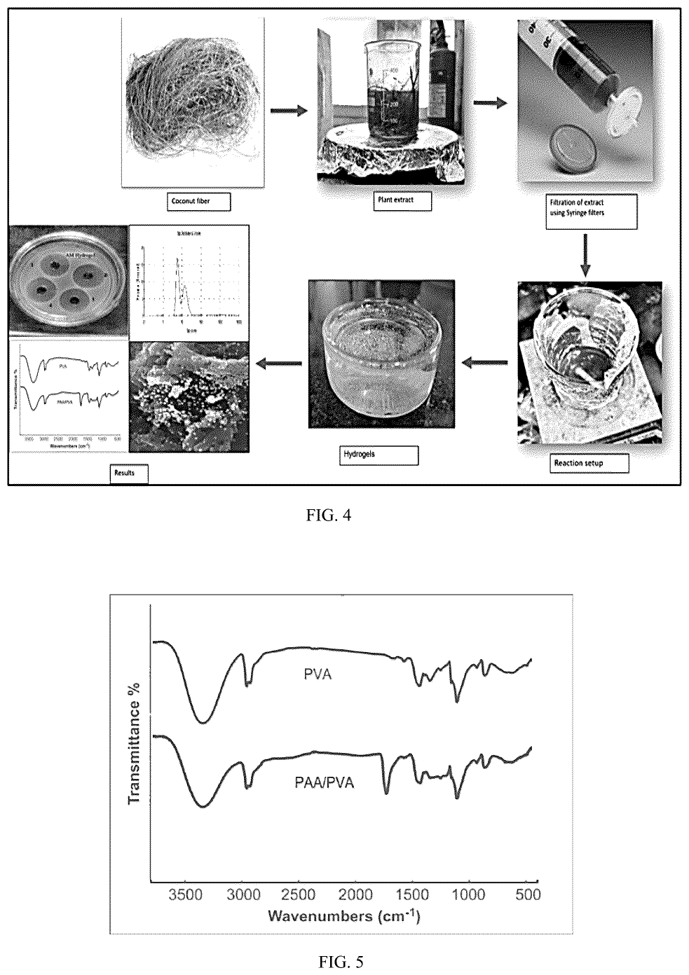

FIG. 4 shows Schematic illustration of Synthesis of Ag—N PVA/PAA hydrogel.

The synthesis of Ag—Ni bimetallic nanoparticles was achieved by combining 12.5 ml of 12 mM AgNO3 with 12.5 ml of 9 mM Ni (NO3)3 in a PVA/AA matrix. After that, the tube was stirred while being heated to a temperature of 60° C. As soon as this temperature was attained, 6 mL of the filtered coconut coir extract was added to the mixture. The mixture was then maintained on a steady stir and heated to 80° C. for one hour. The transformation of the liquid mixture from translucent to light brown served as a monitoring and confirmation mechanism for the creation of the nanoparticles throughout the process.

Given Below are the Primary Goals of this Invention:
a) One-pot synthesis of Ag—Ni-PVA/PAA hydrogel using coconut coir extract
b) To study the antimicrobial activity of synthesized Ag—Ni-PVA/PAA hydrogel against microbes developed on a wound.

### 3. Results and Discussion

### 1. FT-IR Spectra

FIG. 5 shows FT-IR spectra of Ag—Ni-PVA/PAA hydrogel.

The C═O stretching band of PAA at 1711 cm-1 was introduced and softly moved towards the lower wave numbers as a result of the addition of PAA to PVA, while the C—O stretching vibration of pure PVA at 1241 cm-1 was progressively strengthened and widened. This demonstrates the formation of a new intermolecular hydrogen bond interaction and verifies that those between PVA and PAA have replaced the PVA hydrogel's hydrogen bond interactions. Also, although the C—O stretching vibration at 1090 cm-1 was somewhat enhanced, the peak of the O—H stretching vibration for the pure PVA membrane, at 3300 cm-1, was progressively expanded and attenuated. This could be brought on by the esterification process between the hydroxyl and carboxylic acid groups in PVA and PAA.

### Clean Copy

### 2. Swelling Experiments

In addition to measuring polymer film swelling ratios in water and a bicarbonate/phosphate-buffered solution (CBS/PBS, pH 7.4), the dry polymer films were submerged in the liquid for 48 hours prior to being weighed in order for the swelling to become close to equilibrium at room temperature. The free liquid that had gathered on top of the inflated films was then promptly wiped-out using filter paper before the sample weights were once again measured. The abbreviation SR stands for the swelling ratio of the films, and it is described as follows:

$$SR = ( {M}_{t} - {M}_{0} ) / {M}_{0} \times 100 \%$$

The swelling results of different Ag—Cu/PVA/PAA hydrogel in pure water and CBS/PBS are 110%. This indicates that hydrogel shows good absorbing properties. Hence, it can help in quick drying of wound by absorbing wound fluid.

### 3. Dynamic Light Scattering (DLS)

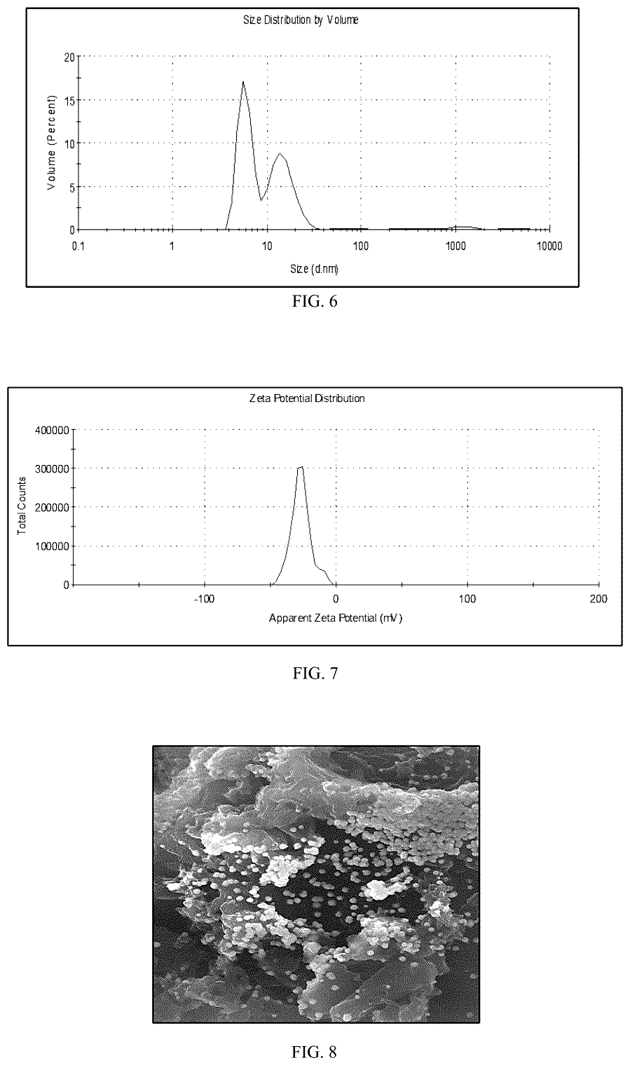

FIG. 6 shows Particle size measurement using DLS.

The size of nanoparticles is measured using dynamic light scattering, and their stability in suspension over time is also assessed.

FIG. 6 shows the size distribution of the prepared nanoparticles. The size distribution and polydispersity index (PDI) of Cu—Ag NPs were detected by DLS analysis. The results have indicated the presence of smaller particles of measures 12 nm (69%) and 9 nm (31%). Z-average size and PDI for the synthesized nanoparticles were observed to be 11.1 nm and 0.3 nm respectively.

### 4. Zeta Potential

FIG. 7 shows the zeta potential distribution for Ag—Ni NP. The zeta potential value for the synthesized nanoparticles was found to be −47 mV.

### 5. SEM Analysis

FIG. 8 shows Ag—Ni PVA/PAA hydrogel SEM image.

It can be clearly seen in FIG. 8 that nanoparticles were successfully synthesized in the polymer matrix.

6. Well Diffusion Method to Test the Antimicrobial Activity of a Compound Against E. coli

### Materials:

E. coli culture, Nutrient agar, Sterile petri plates, Sterile pipettes, Sterile tips, Sterile water, The test compounds, A sterile cork, borer or a sterile pipette tip, Incubator set at 37° C., Ruler Disinfectant solution.

### Procedure:

Sterilize the Petri plates by autoclaving or by exposing them to UV light for at least 30 minutes. Prepare the nutrient agar by following the manufacturer's instructions and pour it into the sterilized petri plates to a depth of 3-4 mm. Allow the agar to solidify completely by leaving it undisturbed for 30-60 minutes at room temperature. Inoculate the agar with E. coli by spreading a loopful of the culture uniformly over the surface of the agar using a sterile loop. Make wells in the agar using a sterile cork borer or a sterile pipette tip. The wells should be of equal size and depth (around 6 mm in diameter and 3 mm in depth) and spaced out evenly. Add the test compound to the well using a sterile pipette. Incubate the plates at 37° C. for 24 hours. After incubation, measure the diameter of the clear zone of inhibition around each well using a ruler. The zone of inhibition is the area around the well where no bacterial growth is observed. Repeat the experiment four times to ensure the accuracy and reproducibility of the results. Calculate the mean diameter of the zone of inhibition and compare it with the control to determine if the test compound has antimicrobial activity against E. coli.

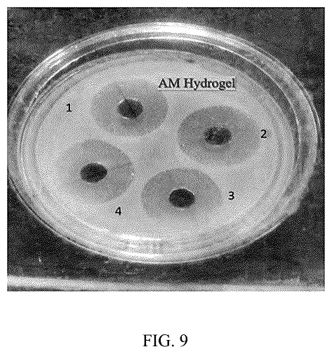

FIG. 9 shows Well diffusion method test image.

Observation Table

| | Inhibitory zone diameter |
|---|---|
| Trial no | to the nearest millimeter (mm) |
| 1 | 23 |
| 2 | 22 |
| 3 | 23 |
| 4 | 23 |
| Average | 22.75 |

### Result:

Average Inhibitory zone diameter to the nearest millimeter (mm) was found to be 22.75 (mm).

What is claimed is:

1. We have successfully synthesized one pot Cu—Ag bimetallic nanoparticles with particle size less than 15 nm by using coconut shell extract.

2. The as synthesized nanoparticles by using green approach are successfully incorporated in medical grade hydrogels.

3. The synthesized hydrogels are showing excellent antimicrobial activities, average inhibitory zone diameter to the nearest millimetre (mm) was found to be 22.75 (mm) and because of this, it can be used to reduce infection, pain relief, and rapid healing.
